(19) United States
(12) Patent Application Publication
(10) Pub. No.: US 2024/0225011 A1
(43) Pub. Date: Jul. 11, 2024

(54) MICROBIAL INOCULANTS FOR PLANT ROOTING CAPACITY AND GERMINATION RATE

(71) Applicant: FULL CIRCLE MICROBES, INC., Hinesburg, VT (US)

(72) Inventors: Victoria I. HOLDEN, Essex Junction, VT (US); Charles SMITH, Essex Junction, VT (US); Katelynn KOSKIE, Essex Junction, VT (US); Rogelio ZIMBRON, Burlington, VT (US); Jack EVANS, Burlington, VT (US); Amy ZIOBRON, Burlington, VT (US)

(21) Appl. No.: 18/559,249

(22) PCT Filed: May 9, 2022
(86) PCT No.: PCT/US2022/028368

§ 371 Date: Nov. 6, 2023

Related U.S. Application Data
(60) Provisional application No. 63/185,682, filed on May 7, 2021.

(51) Int. Cl. A01N 63/27; A01N 63/20; A01N 63/25; A01N 63/30; A01N 63/38; A01P 21/00

(52) U.S. Cl. CPC A01N 63/27; A01N 63/20; A01N 63/25; A01N 63/30; A01N 63/38; A01P 21/00

(57) ABSTRACT

This disclosure describes microbial compositions that contain novel combinations of at least two microbial organisms that, when included in a microbial inoculate and applied to applied to seeds, plants, roots, or growth substrates, promote plant rooting, or if applied to seeds, promote seed germination.

### CROSS REFERENCE TO RELATED APPLICATIONS

This application claims priority to U.S. Provisional Application No. 63/185,682, filed on May 7, 2021, the disclosure of which is incorporated by reference.

### TECHNICAL FIELD

The inventions described herein relates to microbial strains, compositions, and methods that are useful for promoting rooting capacity and/or germination rate.

### BACKGROUND OF THE INVENTION

Worldwide, farmers spend over $376B on fertilizer and agricultural chemicals to stimulate crop yield and protect against pests (1). The application of these chemicals has significant unintended consequences for ecological health, amounting to $157B in damages to the environment and human health in the U.S. alone (2). Pesticide exposure has been linked to higher rates of certain cancers (3) and 700% higher rate of miscarriages and birth defects (4). These harms most frequently impact communities of color, who are exposed to 56-63% more pollution than they produce (5).

Farmers face an economic and practical dilemma in their need to maintain high crop outputs, and they have used the tools available to them. The Agricultural industry requires new solutions to increase crop productivity without chemicals that damage the environment and human health. Optimized microbial communities offer a compelling alternative with the potential to benefit farmers as well as ecological health.

Soil health is a vital component to the lifetime performance of nearly all plants. In addition to providing water and a variety of nutrients essential to plant growth including nitrogen, potassium, and phosphorus, healthy soil also contains gases, organic matter, and microorganisms (6). Estimates from garden and farm soils show that one teaspoon of soil contains up to 1 billion bacteria and numerous yards of fungi (7).

Bacterial and fungal microorganisms living in the soil can act as decomposers participating in nutrient cycling, as mutualists forming beneficial relationships with plants, or as pathogens causing disease in the plants they infect (8,9). Bacterial mutualists are found throughout the soil, but are highly concentrated in the rhizosphere, the narrow region of soil next to and inside plant roots (10). Within the rhizosphere, roots release exudates to stimulate the growth of beneficial bacteria (10). Once established, these bacteria will promote plant growth by suppressing pathogens, converting nutrients like atmospheric nitrogen to bioavailable forms for plant uptake, and producing growth-promoting compounds like phytohormones (10).

The most common fungal mutualists are mycorrhizal fungi (10). Mycorrhizal fungi can live on the surface of roots (ectomycorrhizal) or inside roots (endomycorrhizal or arbuscular mycorrhizal) where they provide the root with access to water and nutrients in exchange for photosynthetically-derived carbohydrates (10). Mycorrhizal fungi can also suppress the growth of plant pathogens (11).

Certain mycorrhizal fungi and other beneficial microbes, including various bacterial species may be useful for resolving problems currently facing the agricultural industry regarding current fertilization practices. The introduction of mycorrhizal fungi and other beneficial microbes can positively impact plants of industrial crops and home gardens alike. These plants include hops, ornamental flowers (roses, orchids, lavender, lilies, geranium, marigold), saffron, Cannabis, Christmas trees (fir trees), wine grapes, sunflowers, broccoli, rice, tomatoes, sugar cane, corn, wheat, soy, cotton, and tea (C. sinensis var. sinensis and C. s. var. assamica).

Cannabis represents a highly attractive use-case for alternatives to traditional fertilizers. For example, the increasing use of clones as a means of propagation in the Cannabis industry has prompted the need for rapid and robust rooting outcomes. Microorganisms like bacteria and fungi have been shown to increase root biomass and alter root architecture (12-14). In addition, a large quantity of Cannabis seeds is sold every year in the U.S. Combining seeds with beneficial microbes has the potential to increase germination and seedling growth metrics. (15, 16).

### SUMMARY OF THE INVENTION

This disclosure describes embodiments of microbial compositions for promoting plant rooting or seed germination. In various embodiments, the microbial compositions contain a mixture of: at least one first microbial species selected from Azospirillum brasilense, Sphingomonas paucimobilis, Rhizophagus irregularis, Bradyrhizobium japonicum, Paenibacillus mucilaginosus, Pseudomonas putida, Variovorax paradoxus, Rhizophagus intraradices, Rhizophagus diaphanus, Rhizophagus clarus, Saccharomyces cerevisiae, and Trichoderma harzianum; and at least one second microbial species selected from Pseudomonas fluorescens, Azotobacter chroococcum, Herbaspirillum seropedicae, Paenibacillus lentimorbus, Azorhizobium caulinodans, Gluconacetobacter diazotrophicus, Trichoderma hamatum, Trichoderma virens, Beijerinckia mobilis, and Laccaria bicolor. For example, in some embodiments, a microbial composition for promoting plant rooting or seed contains at least one first microbial species selected from Azospirillum brasilense, Rhizophagus irregularis, Paenibacillus mucilaginosus, Pseudomonas putida, and Rhizophagus intraradices, and at least one second microbial species is selected from Azotobacter chroococcum, Herbaspirillum seropedicae, Paenibacillus lentimorbus, Beijerinckia mobilis, and Trichoderma hamatum.

In embodiments of a microbial composition of the invention that promotes plant rooting capacity, the composition contains:
a) A. brasilense, P. putida, G. diazotrophicus, T. virens, and P. fluorescens;
b) A. brasilense, P. putida, G. diazotrophicus, T. virens, P. fluorescens, and S. cerevisiae;
c) P. putida, A. brasilense, P. lentimorbus, and T. virens;
d) G. diazotrophicus, A. caulinodans, P. fluorescens, V. paradoxus, and T. virens; or
e) P. putida, A. brasilense, P. lentimorbus, T. virens, G. diazotrophicus, A. caulinodans, P. fluorescens, and V. paradoxus.

In other embodiments in which a microbial composition of the invention promotes seed germination, the microbial composition contains:
a) A. brasilense, P. putida, and A. chroococcum; or
b) A. brasilense, P. putida, A. chroococcum, and B. mobilis.

In some embodiments of the invention, a microbial composition of the invention is lyophilized and resuspended in water or an aqueous solution to form a microbial inoculant, which may, optionally, include a carbon source and/or a thickening agent.

### BRIEF DESCRIPTION OF THE FIGURES

FIG. 1 depicts average shoot length of sunflower seedlings 7 days after treatment with a rooting inoculant in an outdoor field.

FIG. 2 depicts average branching zone length of sunflower seedlings 4 days after treatment with a rooting inoculant in an outdoor field.

FIG. 3 depicts average total mass of sunflower seedlings 4 days after treatment with a rooting inoculant in an outdoor field.

FIG. 4 depicts average crown diameter of sunflower seedlings 4 days after treatment with a rooting inoculant in an outdoor field.

FIG. 5A depicts average number of lateral roots of sunflower seedlings 4 days after treatment with a rooting inoculant in an outdoor field.

FIG. 5B depicts average number of lateral roots of sunflower seedlings 7 days after treatment with a rooting inoculant in an outdoor field.

FIG. 6 depicts average plant growth index (PGI) of sunflower plants treated with a rooting inoculant in an outdoor field.

FIG. 7 depicts rooting percentage of hemp clones treated with a rooting inoculant.

FIG. 8 depicts average number of days to root emergence of hemp clones treated with a rooting inoculant.

FIG. 9 depicts average number of roots of hemp clones 7 days after treatment with a rooting inoculant.

FIG. 10 depicts average number of roots of hemp clones 14 days after treatment with a rooting inoculant.

FIG. 11 depicts average shoot length of hemp seedlings 4 days after treatment with a rooting inoculant.

FIG. 12A depicts average root length of hemp seedlings 4 days after treatment with a rooting inoculant.

FIG. 12B depicts average root length of hemp seedlings 7 days after treatment with a rooting inoculant.

FIG. 13A depicts average branching zone length of hemp seedlings 4 days after treatment with a rooting inoculant.

FIG. 13B depicts average branching zone length of hemp seedlings 7 days after treatment with a rooting inoculant.

FIG. 14A depicts average number of lateral branches of hemp seedlings 4 days after treatment with a rooting inoculant.

FIG. 14B depicts average number of lateral branches of hemp seedlings 7 days after treatment with a rooting inoculant.

FIG. 15 depicts average total weight of hemp seedlings 4 days after treatment with a rooting inoculant.

FIG. 16 depicts average shoot length of hemp seedlings treated with a rooting inoculant.

FIG. 17 depicts average root length of hemp seedlings treated with a rooting inoculant.

FIG. 18 depicts average branching zone length of hemp seedlings treated with a rooting inoculant.

FIG. 19 depicts average number of lateral roots of hemp seedlings treated with a rooting inoculant.

FIG. 20 depicts average total weight of hemp seedlings treated with a rooting inoculant.

FIG. 21 depicts average root weight of hemp seedlings treated with a rooting inoculant.

FIG. 22A depicts average shoot length of hemp seedlings 4 days after treatment with a rooting inoculant.

FIG. 22B depicts average shoot length of hemp seedlings 7 days after treatment with a rooting inoculant.

FIG. 23 depicts average shoot length of corn seedlings treated with a rooting inoculant.

FIG. 24 depicts average primary root length of corn seedlings treated with a rooting inoculant.

FIG. 25 depicts average number of roots of corn seedlings treated with a rooting inoculant.

FIG. 26 depicts average branching zone length of corn seedlings treated with a rooting inoculant.

FIG. 27 depicts average number of lateral branches of corn seedlings treated with a rooting inoculant.

FIG. 28 depicts average total weight of corn seedlings treated with a rooting inoculant.

FIG. 29 depicts average root weight of corn seedlings treated with a rooting inoculant.

FIG. 30 depicts indole acetic acid (IAA) levels measured in rooting inoculants supplemented with exogenous tryptophan.

FIG. 31 depicts average germination percentage of corn seeds treated with a germination inoculant.

FIG. 32 depicts average germination percentage of cosmos seeds treated with a germination inoculant.

FIG. 33 depicts average germination percentage of legume seeds treated with a germination inoculant.

FIG. 34 depicts average germination percentage of sunflower seeds treated with a germination inoculant.

FIG. 35 depicts average hemp biomass of hemp plants treated with a germination inoculant.

FIG. 36 depicts average leaf surface area of legume plants treated with a germination inoculant.

FIG. 37 depicts average biomass of legume plants treated with a germination inoculant.

### DETAILED DESCRIPTION

The inventions described herein relate to microbial compositions that contain combinations of microbial species, which when applied to seeds, plants, roots, or growth substrates (soils, hydroponic media, aeroponic media, etc.), promote certain desirable characteristics. A microbial composition of the invention may comprise, consist essentially of, or consist of a mixture of microbial species as described here. A microbial composition of the invention may have mixtures of microbial species that are particularly suited for promoting plant rooting capacity. While another microbial composition of the invention promotes germination rate. In yet another microbial composition of the invention promotes plant rooting capacity and promotes germination rate. A microbial composition of microbial species may also be a lyophilized microbial composition of the microbial species.

Typically, microbial compositions of the invention promote plant germination and/or rooting by: (i) increasing the bioavailability of nutrients for uptake by the plants; (ii) altering the production or activity of plant hormones; or (iii) a combination of (i) and (ii). More generally, microbial compositions of the invention promote plant germination and/or rooting capacity by beneficially interacting with the plants.

A microbial composition or a microbial inoculant having the first and second microbial species as discussed above increases or promotes rooting capacity through the uptake of additional nutrients, plant hormone alterations, and the alteration of microbial diversity. Indication of rooting capacity can pertain to root growth rate, root size, number of roots produced, and root architecture. Root growth rate can be quantified by the millimeters of growth per a set amount of time. Root size measurements can be quantified by root length and root diameter. Root architecture can be measured by mapping and observing root architecture traits such as root number, angles of roots, and branching pattern. Microbial diversity can be quantified through next-generation sequencing techniques.

Furthermore, a microbial composition or a microbial inoculant having the first and second microbial species as discussed above increases or promotes germination rate by increasing the bioavailability of nutrients, altering hormones that promote plant growth and/or germination, and altering the microbial composition of the plant and/or seed microbiome. Seed germination can be quantified by comparison of the germination rate of a plant species under experimental versus controlled conditions, where germination rate is defined as the average number or average percentage of seeds germinating per period of time.

Typically, microbial compositions of the invention promote rooting capacity and germination rate, ultimately in order to increase yield. And increases in yield, a term that is generally understood in the agricultural industry to mean sellable yield, may correspond to an increased harvest yield or increased plant biomass.

As disclosed above, a microbial composition of the invention contains a specified mixture of microbial species—typically fungal and bacterial species. For example, in certain microbial compositions of the invention, one or more species of a mycorrhizal fungi may be combined with: (i) another fungal species, including with one or more other species of mycorrhizal fungi; (ii) one or more bacterial species of bacteria; or (iii) a combination of fungal and bacterial species.

The microbial species of a microbial composition of the invention may have, but not necessarily, been isolated prior to being mixed into a composition with each other. More specifically, an isolated microbial species has been removed from its natural or culture milieu. Though, “isolated” does not necessarily reflect the extent to which the microbe has been purified.

In certain microbial compositions of the invention, the mixture of microbial species includes at least microbial species—a first microbial species—selected from Azospirillum brasilense, Sphingomonas paucimobilis, Rhizophagus irregularis, Bradyrhizobium japonicum, Paenibacillus mucilaginosus, Pseudomonas putida, Variovorax paradoxus, Rhizophagus intraradices, Rhizophagus diaphanus, Rhizophagus clarus, Saccharomyces cerevisiae, and Trichoderma harzianum; and at least one microbial species—a second microbial species—selected from Pseudomonas fluorescens, Azotobacter chroococcum, Herbaspirillum seropedicae, Paenibacillus lentimorbus, Azorhizobium caulinodans, Gluconacetobacter diazotrophicus, Trichoderma hamatum, Trichoderma virens, Beijerinckia mobilis, and Laccaria bicolor.

For example, a particular microbial compositions of the invention useful for enhancing plant rooting capacity and/or germination rate may contain at least one first microbial species selected from Azospirillum brasilense, Rhizophagus irregularis, Paenibacillus mucilaginosus, Pseudomonas putida, and Rhizophagus intraradices that is combined with at least one second microbial species selected from Azotobacter chroococcum, Herbaspirillum seropedicae, Paenibacillus lentimorbus, Beijerinckia mobilis, and Trichoderma hamatum.

Indeed, in a preferred microbial composition of the invention for promoting rooting capacity, the composition contains (i.e., comprises) A. brasilense, P. putida, G. diazotrophicus, T. virens, and P. fluorescens. Another preferred microbial composition according to the invention for promoting rooting capacity contains (i.e., comprises) A. brasilense, P. putida, G. diazotrophicus, T. virens, P. fluorescens, and S. cerevisiae. Another preferred microbial composition according to the invention for promoting rooting capacity contains (i.e., comprises) P. putida, A. brasilense, P. lentimorbus, and T. virens. Another preferred microbial composition according to the invention for promoting rooting capacity contains (i.e., comprises) G. diazotrophicus, A. caulinodans, P. fluorescens, V. paradoxus, and T. virens. Another preferred microbial composition according to the invention for promoting rooting capacity contains (i.e., comprises) P. putida, A. brasilense, P. lentimorbus, T. virens, G. diazotrophicus, A. caulinodans, P. fluorescens, and V. paradoxus. Similarly, a microbial composition of the invention may consist of, or consist essentially of (A. brasilense, P. putida, G. diazotrophicus, T. virens, and P. fluorescens), (A. brasilense, P. putida, G. diazotrophicus, T. virens, P. fluorescens, and S. cerevisiae), (P. putida, A. brasilense, P. lentimorbus, and T. virens), (G. diazotrophicus, A. caulinodans, P. fluorescens, V. paradoxus, and T. virens), or (P. putida, A. brasilense, P. lentimorbus, T. virens, G. diazotrophicus, A. caulinodans, P. fluorescens, and V. paradoxus).

Alternatively, in a preferred microbial composition of the invention for promoting germination rate, the composition contains (i.e., comprises) A. brasilense, P. putida, and A. chroococcum. Another preferred microbial composition according to the invention for promoting germination rate contains (i.e., comprises A. brasilense, P. putida, A. chroococcum, and B. mobilis. Similarly, a microbial composition of the invention may consist of, or consist essentially of (A. brasilense, P. putida, and A. chroococcum) or (A. brasilense, P. putida, A. chroococcum, and B. mobilis).

The microbial species used in a microbial composition or a microbial inoculant of the invention themselves can originate from a frozen glycerol stock, a solid-medium growth plate, or a commercially available source. A microbial species can also be isolated from environmental samples or purchased from open-access culture collections. The selected microbial species can then be streak-plated in a sterile environment on a petri dish or other containers of solid media to generate single colony isolates. Streak-plated samples on petri dishes or other containers can be incubated and isolated using techniques known in the art. For example, a microbial species may be incubated for 24-48 hours or longer at 30° C. aerobically, at 37° C. under normal atmospheric conditions, or at any other condition optimal or sufficient for colony formation for a given species or strain. After incubation and colony formation, individual colonies can be isolated for propagation in liquid media for a further 24-48 hours or longer as stated above. Isolated microbial species or strains can be stored at −80° C. in 25-50% glycerol for continued propagation. Media and growth conditions can vary and are preferably optimized for a given strain. Media can be used for culturing, isolating, and storing microbes. Suitable media can be comprised of a carbon source, an amino acid source, salts, buffers, and yeast or meat extracts. Media can be prepared as a liquid or as a solid by supplementing with agar.

The microbial species used in a microbial composition or a microbial inoculant of the invention can be cultured in media comprising exogenous tryptophan. Some microbial species used in a microbial composition or a microbial inoculant of the invention are capable of enzymatically converting tryptophan to indoleacetic acid (IAA)—a plant hormone capable of increasing rooting capacity and promoting root growth. Accordingly, the presence of exogenous tryptophan in growth media can lead to increased production of IAA, thereby increasing capacity and promoting root growth in plants to which the mixture of microbial species or microbial inoculant are applied.

In a microbial composition or a microbial inoculant of the invention, individual strains or species can be present in equal concentrations. Alternatively, individual strains or species can be present in >1- to 1,000-fold excess over another strain or species present, in >1- to 500-fold excess, in >1- to 100-fold excess, in >1- to 50-fold excess or in >1- to 10-fold excess.

As discussed above a microbial composition containing a mixture of microbial species according to the invention may be a mixture of individually lyophilized microbial species. As known in the art lyophilization is a process by which water is removed by freezing the material and then reducing the pressure and adding heat to allow the frozen water in the material to sublimate. Lyophilization can be used to preserve perishable material, including microbes, and make it more convenient for transport. Preparation of the lyophilized mixture can be accomplished by inoculating, growing, pelleting, and lyophilizing individual species or strains before combining the lyophilized materials to form the lyophilized mixture. Strains of the same species can be combined after pelleting and before lyophilization, or after pelleting and lyophilization.

Starter cultures for lyophilization mixture can be prepared by inoculating a strain from a frozen glycerol stock or solid growth plate into liquid media for example using 5-500 mL volume or other volumes known in the art. Likewise, starter culture volume can be for example <5 mL or >500 mL or other volumes known in the art. Starter cultures can be used to inoculate a bulk culture that is for example 20-50L in volume or other volumes known in the art. Likewise bulk culture can be for example <20 L or >50 L or other volumes known in the art. Bulk culture can be cycled through multiple draw/fill cycles as desired. Draw/fill cycles involve growing the culture to the desired cell density, removing a portion of the culture, and supplementing the remainder with fresh media for continued growth. Once desired cell density is reached, microbes can be pelleted from media by centrifugation. Strains of the same species can be optionally combined, and pellets can be resuspended in, for example, 2 L of media and lyophilized. Resuspension volume can be resuspended in for example volumes <2L or >2 L or volumes depending on the capacity of lyophilization equipment. Individual lyophilized microbial species can be combined to generate the final lyophilized mixture. Lyophilized mixture can be packaged in packets for subsequent distribution and use.

The invention also relates to microbial inoculants. As understood herein, a microbial inoculant contains, at minimum, suspension of a microbial composition of the invention in water or an aqueous solution.

Thus, an inoculant of the invention may be, for example, prepared by resuspending a microbial composition in water. Resuspension volume can be for example <1 gallon (U.S. customary Units equivalent to 3.785 L) or >1 gallon or other volume, such as, but not limited to 200 ml, 250 ml, 300 ml, 350 ml, 400 ml, 450 ml, 500 ml, 550 ml, 600 ml, 650 ml, 700 ml, 750 ml, 800 ml, 850 ml, 1000 ml, 1250 ml, 1500 ml, 1750 ml, 2000 ml, 2250 ml, 2500 ml, 2750 ml, 3000 ml, 3250 ml, 3500 ml, 3750 ml, 4000 ml, or any volume therein.

Optionally, a microbial inoculant of the invention may also contain a carbon source. In other words, a microbial inoculant of the invention may be supplemented with one or more carbon sources. The carbon source of an inoculant of the invention may be admixed with a lyophilized microbial composition or it may be added at the time a lyophilized microbial composition is resuspended in water or an aqueous solution. Examples of carbon sources include, but are not limited to, hexoses, such as glucose, but other sources that are readily assimilated, such as amino acids, may also serve as a carbon source. The amount of a carbon source in a microbial inoculant of the invention may vary depending on the particular combination of microbial species in a microbial composition of the invention; accordingly, the invention does not specify a limit on the total amount of a carbon in a microbial inoculant of the invention. However, in one microbial inoculant of the invention, a carbon source, when present, may constitute 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of microbial inoculant (weight/volume). Accordingly, the amount of a carbon source in some microbial inoculants of the invention be from 9-15%, 10-14%, or 11-13% (w/v).

Optionally, a microbial inoculant of the invention may also contain a thickening agent, such as, but not limited to aloe vera, aloe vera flakes, willow bark extract, silica, pectin, psyllium husk, or a gelling agent. A thickened microbial inoculant can be more easily applied to a cutting or roots thereof. The amount of a thickening agent in a microbial inoculant of the invention may vary depending on the particular use of the inoculant; accordingly, the invention does not specify a limit on the total amount of a thickening agent in a microbial inoculant of the invention. However, in one microbial inoculant of the invention, a thickening agent, when present, may constitute 1%, 2%, 3%, 4%, 5%, 6%, 7%, 8%, 9%, 10%, 11%, 12%, 13%, 14%, 15%, 16%, 17%, 18%, 19%, or 20% of microbial inoculant (weight/volume). Accordingly, the amount of a carbon source in some microbial inoculants of the invention be from 2-8%, 3-7%, or 4-6% (w/v).

Once prepared, microbial inoculants can be poured, sprayed, or otherwise applied to ungerminated seeds, germinated seeds, seedlings, cuttings, roots, maturing or young plants, maturing plants at a time before or at the time of flowering, or a growth substrate in either a greenhouse, field, hydroponic grow space, indoor grow space, aeroponic grow space, or other grow space. Microbial inoculants can be applied to seeds before planting by bathing the seeds in the liquid form. Microbial inoculants can be applied to the germinated seed upon planting by inoculating the growth substrate with the liquid form. The growth substrate can be a mixture comprising at least one of soil, perlite, coco coir, vermiculite, pumice, peat moss, compost, aqueous growth media, rock wood, and worm casting or other growth substrates known in the art. Microbial inoculants in liquid or spray form can be applied to the seedling, cutting, or roots before or at the time of transplanting or propagation. Transplanting can be uprooting and transferring the seedling from one growth substrate or grow space to another. Microbial inoculants can be applied to young or maturing plants as a foliar spray or as a liquid applied to the growth substrate or directly to the roots and/or aerial (e.g. above soil) plant tissue. Microbial inoculants can be applied to the mature plant at time of flowering as a foliar spray or as a liquid applied to the growth substrate or directly to the roots. Microbial inoculants can be applied to mature plants prior to harvest as a foliar spray or as a liquid applied to the growth substrate. Microbial inoculants can be applied to the soil post-harvest in liquid or spray form to inoculate or “prime” the soil or other growth substrate for the next growing season. Priming the soil or other growth substrate allows for microbial communities to be established prior to planting.

Microbial inoculants can be applied as described repeatedly. Application of the microbial inoculants can occur seasonally, bimonthly, monthly, every 3 weeks, every 2 weeks, weekly, every 5 days, every 3 days, daily, or a similar such period of time appropriate for the grow space and plant species.

In certain instances, the concentration of microbes in a repeatedly applied inoculant can differ from that of the originally applied inoculant. Concentration of microbes in a repeated inoculant can differ relative to that of a previously applied inoculant by a factor of >1 to 1,000, by a factor of >1 to 500, by a factor of >1 to 100, by a factor of >1 to 50, by a factor of >1 to 10, or by a factor <1.

### EXAMPLES

### Example 1: Lyophilized Microbial Mixture Production—General Protocol

Bacterial and/or fungal strains are inoculated from frozen glycerol stock or solid growth plate into 5-500 mL liquid media appropriate for each strain. Strains are inoculated from 5-500 mL liquid culture into 20-50 L liquid culture. Cultures are cycled through draw/fill growth cycles as desired. Strains are pelleted by centrifugation and combined into a single 2 L media suspension. The combined 2 L suspension is lyophilized. These steps are repeated for each individual microbial species that is to comprise the inoculant. All lyophilized species are combined to generate a final lyophilized microbial composition. Final concentrations of inoculant species are equal compared to one another and at least optical density at 600 nm (OD₆₀₀)=0.7 (0.7×10⁸ CFU/mL). Lyophilized inoculant is packaged as packets for subsequent use.

Media for each microbe can be comprised of the components as described in Table 1, or can differ according to best practice known in the art.

TABLE 1
Media compositions

| Microbes | Media Name | Media Components |
|---|---|---|
| Pseudomonas putida | Nutrient | Beef extract |
| Sphingomonas paucimobilis | | |
| Variovorax paradoxus | | |
| Pseudomonas fluorescens | | Peptone |
| Azorhizobium caulinodans | | |
| Azospirillum brasilense | | |
| Azotobacter chroococcum | Nitrogen-free | K₂HPO₄ |
| | | MgSO₄•7H₂0 |
| | | CaCO₃ |
| | | NaCl |
| | | FeSO₄•7H₂0 |
| | | NaMO₄•2H₂0 |
| | | Glucose |
| Herbaspirillum seropedicae | Spirillum nitrogen-fixing | KH₂PO₄ |
| | | K₂HPO₄ |
| | | MgSO₄•7H₂0 |
| | | NaCl |
| | | CaCl₂ |
| | medium | FeCl₃ |
| | | Na₂MoO₄•2H₂0 |
| | | Sodium malate |
| | | Yeast extract |
| Paenibacillus lentimorbus | Bacillus Lentimorbus Media | Mueller-Hinton broth |
| | | Yeast extract |
| | | K₂HPO₄ |
| | | Glucose |
| | | Sodium pyruvate |
| Beijerinckia mobilis | Potato Dextrose | Potato infusion |
| Trichoderma hamatum | | Glucose |
| Trichoderma virens | | |
| Trichoderma harzianum | | |
| Bradyrhizobium japonicum | Rhizobium Medium | Yeast extract |
| | | Mannitol |
| | | Air-dried garden soil |
| | | Na₂CO₃ |
| Gluconacetobacter | Sabouraud Glucose | SABOURAUD-2% Glucose- |
| diazotrophicus | | Bouillon |
| Paenibacillus mucilaginosus | Bacillus Mucilaginosus Media | Sucrose |
| | | K₂HPO₄ |
| | | MgSO₄•7H₂0 |
| | | CaCO₃ |
| Rhizophagus diaphanus | Soil/Sand Culture | Root tissue |
| Rhizophagus intraradices | | Soil |
| Rhizophagus clarus | | Sand |
| Rhizophagus irregularis | | |
| Trichoderma hamatum | | |
| Trichoderma virens | | |
| Trichoderma harzianum | | |
| Laccaria bicolor | | |
| Rhizophagus diaphanus | MSR | MgSO₄•7H₂0 |
| Rhizophagus intraradices | | KNO₃ |
| Rhizophagus clarus | | KCl |
| Rhizophagus irregularis | | KH₂PO₄ |
| | | Ca(NO₃)₂•4H₂0 |
| | | Panthotenate Ca |
| | | Biotine |
| | | Nicotinic acid |
| | | Pyridoxine |
| | | Thiamine |
| | | Cyanocobalamine |
| | | Na Fe EDTA |
| | | MnSO₄•4H₂0 |
| | | ZnSO₄•7H₂0 |
| | | H₃BO₃ |
| | | CuSO₄•5H₂0 |
| | | Na₂MoO₄•2H₂0 |
| | | (NH₄)₆Mo₇O₂₄•4H₂0 |
| | | Sucrose |
| Laccaria bicolor | Malt Media, or | malt extract, or |
| | MS Media | Murashige and Skoog powder |

### Example 2: Preparation of a Microbial Inoculant

Lyophilized packet(s) is resuspended in 1 gallon of water or a volume depending upon the area to be inoculated. Optionally, this suspension is supplemented with a sugar and/or carbon source. This forms the “microbial inoculant” for direct application methods of the invention.

### Example 3: Direct Application Methods

### 3.1 Application to Ungerminated Seed or Germinated Seed Upon Planting

The microbial inoculant is applied directly to ungerminated or germinated seed planted directly in soil or other growth substrate (either potted or grounded).

### 3.2 Application to Seedling at Transplanting

The microbial inoculant is applied directly to seedlings transplanted into soil or other growth substrate (either potted or grounded).

### 3.3 Application to Cutting

The microbial inoculant is applied directly to the cutting at the cut sites, or into the substrate or container into which the cutting is transplanted.

3.4 Application Directly to Maturing or Young Plants After Planting and/or Before Flowering

The microbial inoculant is sprayed or otherwise applied directly onto the growth substrate in either a greenhouse, field, hydroponic grow space, indoor grow space, aeroponic grow space, or other grow space.

### 3.5 Application Directly to Soil Post-Harvest for Soil “Priming” Purposes

Inoculant is sprayed or otherwise applied directly to the growth substrate either in a greenhouse, field, hydroponic grow space, indoor grow space, aeroponic grow space, or other grow space after crops have been harvested.

### Example 4: Treatment of Sunflower Seeds with Rooting Inoculants

### 4.1 Preparation and Application of Microbial Inoculants

Microbial inoculants were prepared as follows. Microbial species comprising each rooting inoculant were grown individually in liquid culture and combined at an equal ratio based on OD₆₀₀ to create a final liquid culture with an OD₆₀₀=1. The rooting inoculants were comprised of the following microbial species unless otherwise indicated. All: P. putida, A. brasilense, P. lentimorbus, T. virens , G. diazotrophicus , A. caulinodans, P. fluorescens, and V. paradoxus; Formula 2: G. diazotrophicus, A. caulinodans, P. fluorescens, V. paradoxus, and T. virens; Original: P. putida, A. brasilense, P. lentimorbus, and T. virens. Inoculants were diluted by a factor of 10 where indicated.

Sunflower seeds were planted in soil and inoculated by applying rooting inoculants to the soil surface 2 days post-planting unless otherwise indicated.

### 4.2 Results

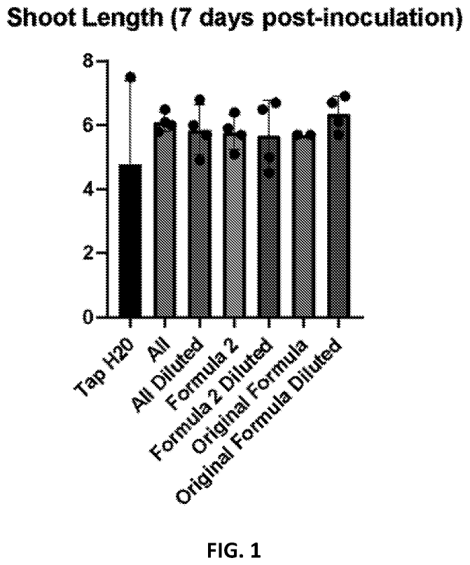

All Root Enhancement inoculants increased the average shoot length of sunflower seedlings compared to water alone (FIG. 1). Each solid bar represents the average shoot length with individual values (n≥2). Measurements were taken 7 days post-inoculation. All treatment groups showed an increase in average shoot length compared to the water-treated control.

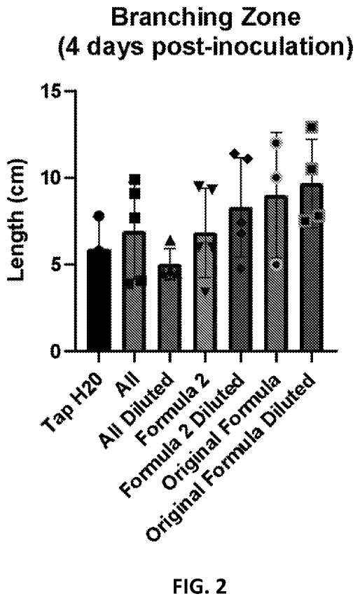

Formula 2 Diluted, Original, and Original Diluted inoculants had to longest branching zone lengths at Day 4 (FIG. 2). Each solid bar represents the average branching zone length with individual values (n≥2). Measurements were taken 4 days post-inoculation. Branching zone length is defined as the length from the seedling crown to the last lateral root branch. All treatments except “All Diluted” increased root branching zone at 4 days post-inoculation.

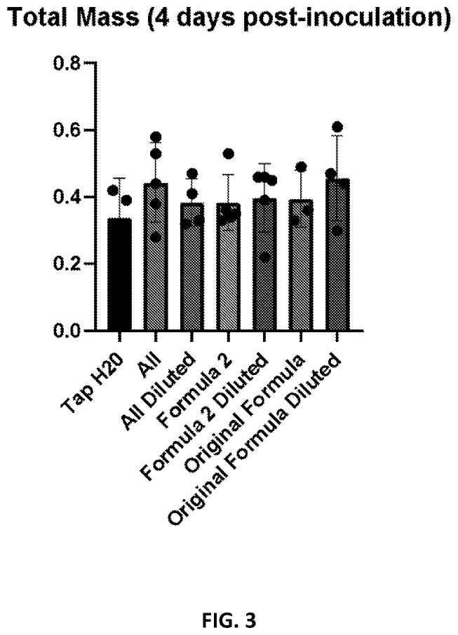

All and Original 1:10 inoculants had the highest average total seedlings masses among the treatment groups (FIG. 3). Each solid bar represents the average total seedling mass with individual values (n≥3). Sunflower seeds were planted in soil and inoculated on the soil surface 2 days post-planting. Measurements were taken 4 days post-inoculation. There was an observable increase in total seedling mass for all treatment groups, with All and Original Diluted inoculants having the largest increase.

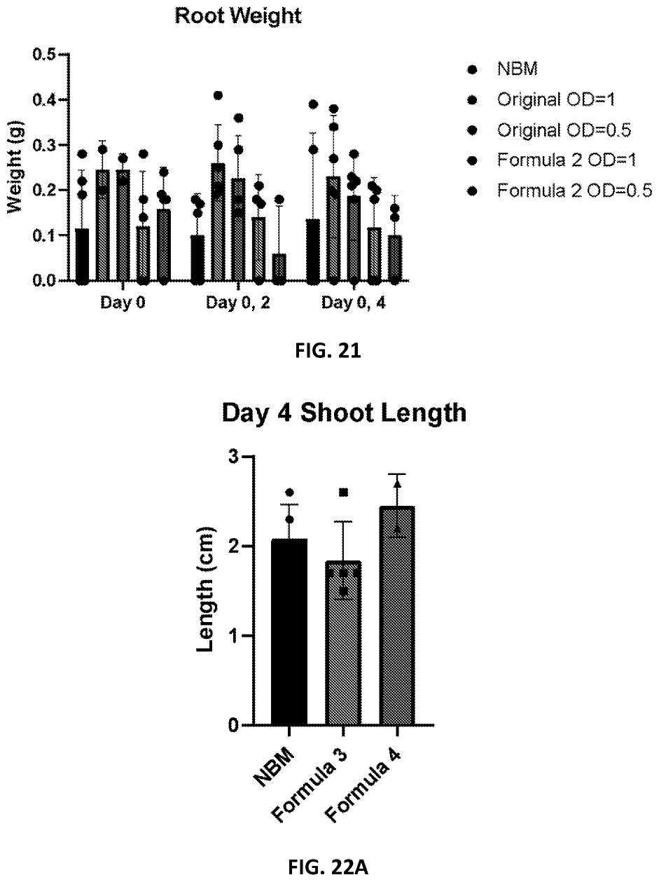

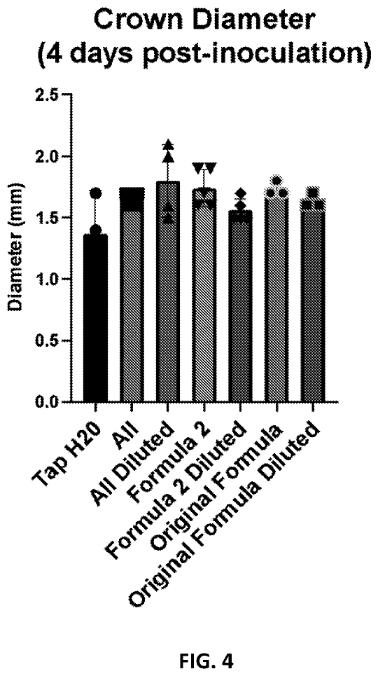

All Root Enhancement inoculants increased sunflower seedling crown diameter at 4-days post-inoculation (FIG. 4). Each solid bar represents the average crown diameter with individual values (n≥2). Measurements were taken 4 days post-inoculation. Crown diameter is defined as the diameter of the seedling where the shoot and root portions of the seedling join. All treatment groups showed an increase in crown diameter 4 days post-inoculation compared to water alone.

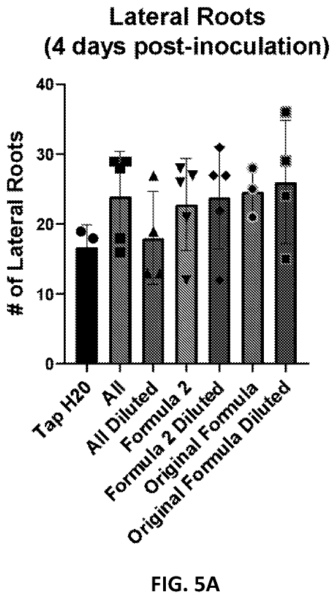

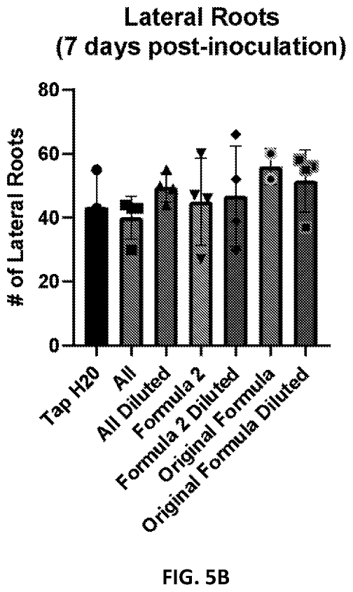

Root Enhancement inoculants increased the number of lateral roots in sunflower seedlings at 4 days post-inoculation (FIGS. 5A and 5B). Each solid bar represents the average number of lateral roots with individual values (n≥2). Measurements were taken 4 days and 7 days post-inoculation. Lateral Roots are defined as secondary root growth extending away from the primary root. All treatment groups showed an increase in the average number of lateral roots 4 days post-inoculation when compared to the water control group (FIG. 5A). All treatment groups except for one, All, showed an increase in the average number of lateral roots at 7 days post-inoculation compared to water alone (FIG. 5B).

Field-grown inoculant-treated sunflowers had greater Plant Growth Indexes compared to untreated plants (FIG. 6). Each solid bar represents the Plant Growth Index (PGI) with individual values (n≥4). Sunflower seeds were sown in soil and inoculated on the soil surface 3 days later. The rooting inoculants were comprised of the following microbial species. Rooting: A. brasilense, P. lentimorbus, P. putida, and T. virens; Rooting + Growth: A. brasilense, P. lentimorbus, P. putida, T. virens, A. chroococcum, B. subtilis, R. palustris, S. paucimobilis, and V. paradoxus. Seedlings were harden-off and transplanted in an outdoor plot 14 days after seeds were sown. Plants received two additional inoculations after transplantation. All treatment groups outperformed untreated plants. PGI is calculated by averaging together each plant's height, width, and depth.

### Example 5: Treatment of Hemp Clones with Rooting Inoculants

### 5.1 Preparation and Application of Microbial Inoculants

Microbial inoculants were prepared as follows. Microbial species comprising each rooting inoculant were grown individually in liquid culture and combined at an equal ratio based on OD₆₀₀ to create a final liquid culture with an OD₆₀₀=1. The rooting inoculants were comprised of the following microbial species. All: P. putida, A. brasilense, P. lentimorbus, T. virens , G. diazotrophicus , A. caulinodans, P. fluorescens, and V. paradoxus; Formula 2: G. diazotrophicus, A. caulinodans, P. fluorescens, V. paradoxus, and T. virens; Original: P. putida, A. brasilense, P. lentimorbus, and T. virens. Hemp clones were either dipped directly into 5X inoculant then place in a water-soaked propagation cube or was dipped then placed in propagation cubes saturated with 1X inoculant.

### 5.2 Results

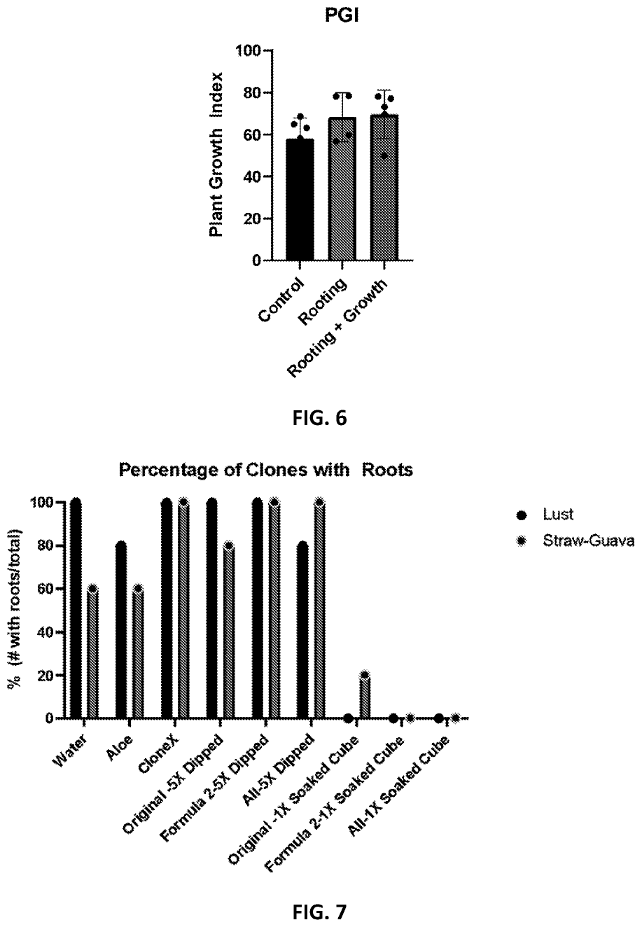

Clones dipped directly in the 5X Root Enhancement inoculants resulted in a similar number of rooted hemp clones as CloneX (FIG. 7). Each solid bar represents the percentage of clones with roots visible on the propagation cubes with individual values (n≥5). The percentage of rooted clones dipped directly into 5X inoculant was comparable to clones dipped in CloneX.

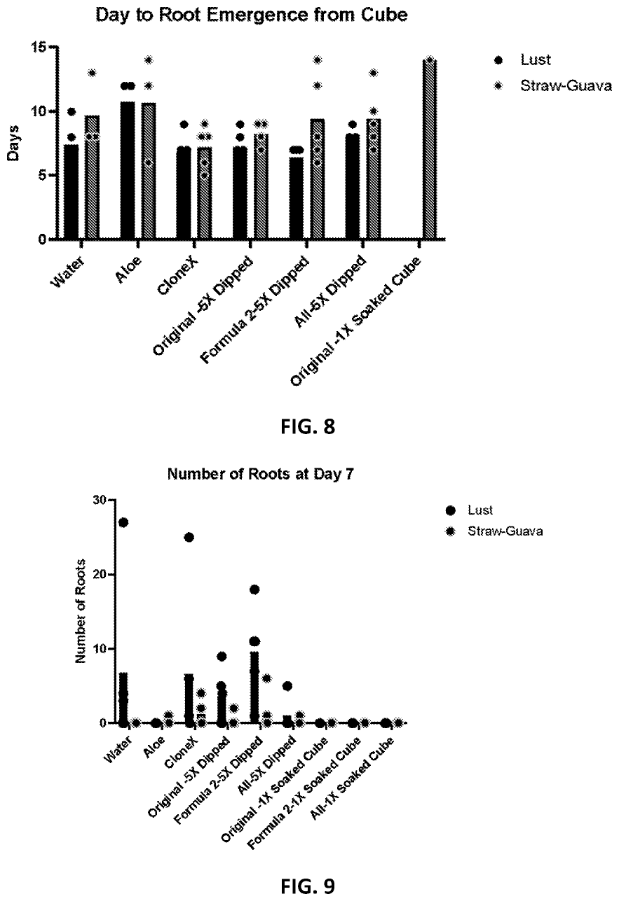

Hemp clones dipped directly in 5X Root Enhancement inoculants resulted in comparable root emergence times as CloneX (FIG. 8). Each solid bar represents the average number of days to root emergence with individual values (n≥5). Clones treated with the 5X inoculants had similar average root emergence times as CloneX.

Lust hemp clones dipped directly in the 5X Original and Formula 2 Root Enhancement inoculants resulted in a similar number of roots as CloneX at day 7 (FIG. 9). Each solid bar represents the average number of days to root emergence with individual values (n≥5). At Day 7, treatment with the 5X Original and Formula 2 inoculants resulted in comparable number of roots to CloneX.

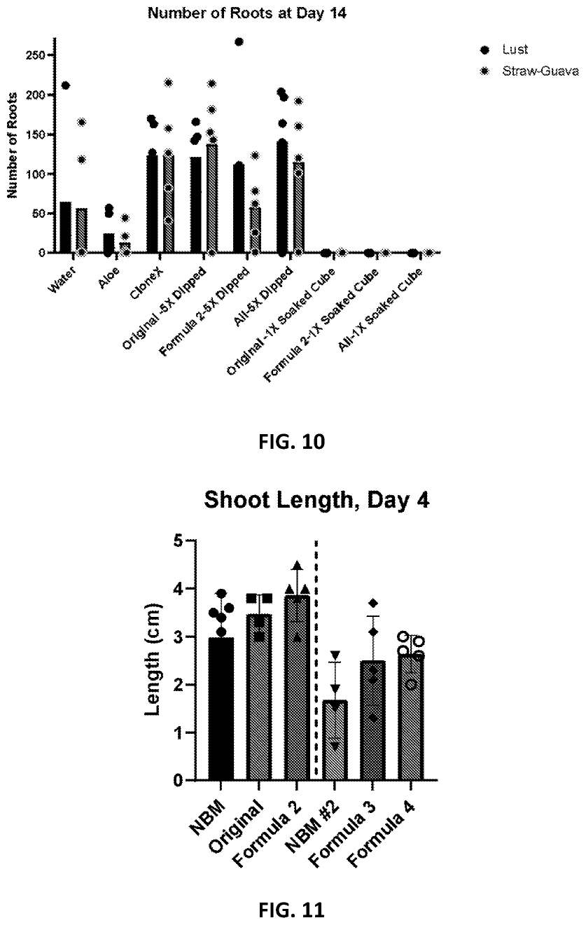

Hemp clones dipped directly in the 5X Root Enhancement inoculants resulted in a similar number of roots as CloneX at day 14 post-inoculation (FIG. 10). Each solid bar represents the average number of days to root emergence with individual values (n≥5). At day 14 post-inoculation, treatment with the 5X Root Enhancement inoculants resulted in comparable numbers of roots to CloneX.

### Example 6: Treatment of Hemp Seeds with Rooting Inoculants

### 6.1 Preparation and Application of Microbial Inoculants

Microbial inoculants were prepared as follows. Microbial species comprising each rooting inoculant were grown individually in liquid culture and combined at an equal ratio based on OD₆₀₀ to create a final liquid culture with an OD₆₀₀=1 unless otherwise indicated. The rooting inoculants were comprised of the following microbial species. Original: P. putida, A. brasilense, and P. lentimorbus; Formula 2: G. diazotrophicus, A. caulinodans, P. fluorescens, and V. paradoxus; Formula 3: P. putida, P. lentimorbus, A. caulinodans, and G. diazotrophicus; Formula 4: A. brasilense, V. paradoxus, P. fluorescens, and G. diazotrophicus. Hemp seeds were soaked in the specified inoculant for 30 minutes then planted immediately unless otherwise indicated.

### 6.2 Results

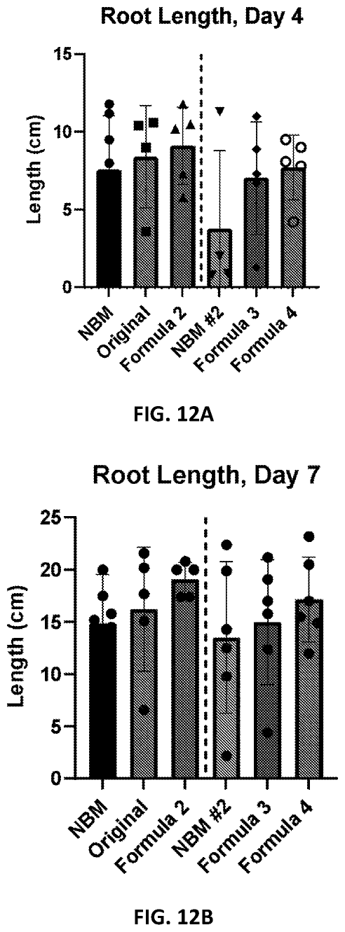

All inoculants increased root length at Day 4 and Day 7 in hemp seedlings (FIGS. 12A and 12B). Each solid bar represents the average root length with individual values (n≥4). Measurements were taken 4 days and 7 days post-inoculation. All inoculants increased the average root length at Day 4 (FIG. 12A) and Day 7 (FIG. 12B) compared the NBM control group. Each of FIGS. 12A and 12B represent two experiments separated by a dashed line. Each experiment was performed using the same methods but were separated by one week.

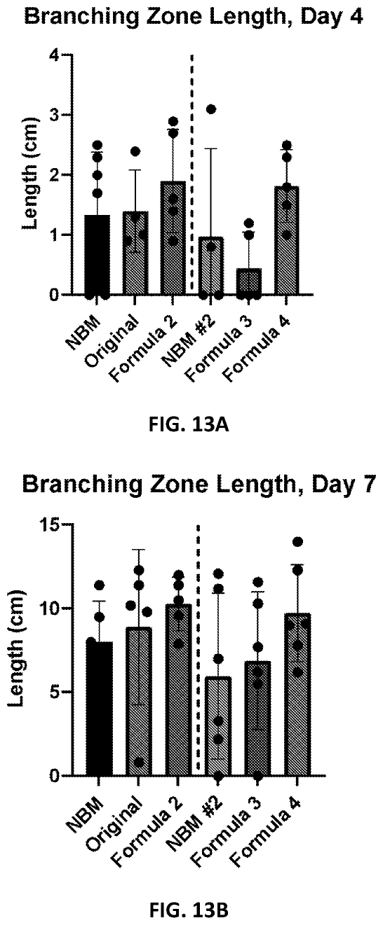

All inoculants increased branching zone length at Day 7 in hemp seedlings (FIGS. 13A and 13B). Each solid bar represents the average shoot length with individual values (n≥4). Measurements were taken 4 days and 7 days post-inoculation. At Day 4, Formula 2 and Formula 4 increased branching zone length (FIG. 13A). All inoculants at Day 7 increased branching zone length comparable to the control (FIG. 13B). Each of FIGS. 13A and 13B represent two experiments separated by a dashed line. Each experiment was performed using the same methods but were separated by one week.

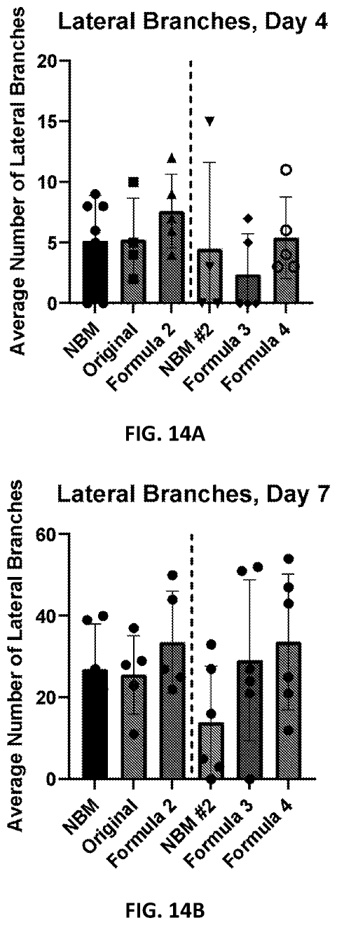

Formula 2, 3, and 4 increased the number of lateral root branches at Day 7 in hemp seedlings (FIGS. 14A and 14B). Each solid bar represents the average number of lateral root branches with individual values (n≥4). Measurements were taken 4 days and 7 days post-inoculation. At Day 4, Formula 2 and Formula 4 increased the average number of lateral branches comparable to the NBM control (FIG. 14A). At Day 7, Formula 2, 3, and 4 increased the number of lateral (FIG. 14B). Each of FIGS. 14A and 14B represent two experiments separated by a dashed line. Each experiment was performed using the same methods but were separated by one week.

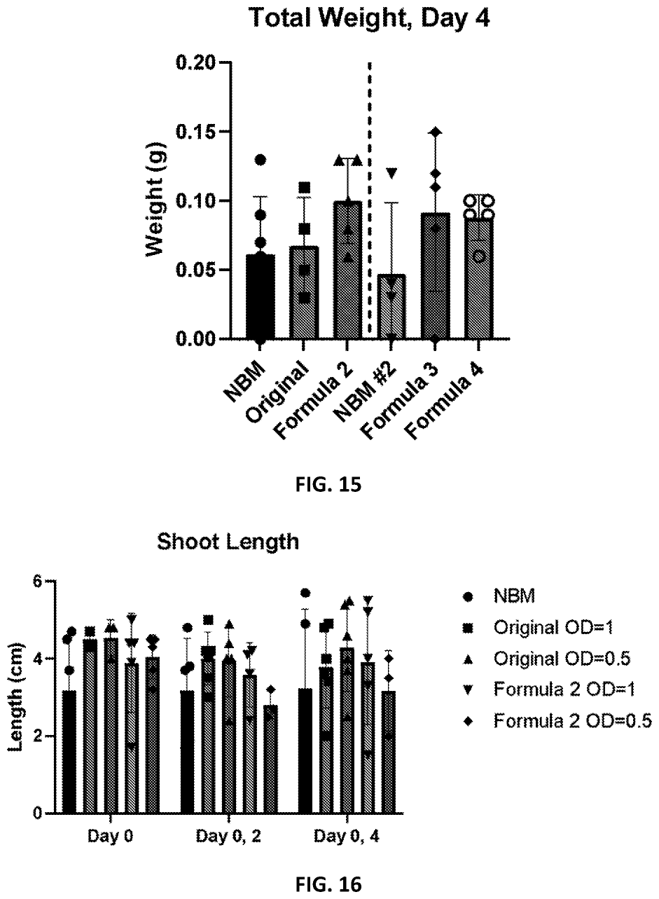

Formulas 2, 3, and 4 increased the total weight of hemp seedlings at Day 4 (FIG. 15). Each solid bar represents the average weight of seedlings with individual values (n≥4). Measurements were taken 4 days post-inoculation. At Day 4, Formulas 2, 3, and 4 increased the average weight of seedlings compared to the NBM control. FIG. 15 represents two experiments separated by a dashed line. Each experiment was performed using the same methods but were separated by one week.

For the following experiments, hemp seeds were sown in soil then inoculated on the soil surface either immediately (Day 0), at Day 0 and at Day 2 (Day 0, 2), or at Day 0 and Day 4 (Day 0, 4) post-sowing unless otherwise indicated. Inoculants were prepared as a final liquid culture with an OD₆₀₀=1 or OD₆₀₀ =0.5 where indicated.

The Original inoculant increased shoot length in hemp seedlings across all application times (FIG. 16). Each solid bar represents the average shoot length with individual values (n≥2). Measurements were taken 7 days post-inoculation. The Original inoculant increased shoot length across all conditions when compared to the NBM control. Both versions of Formula 2 increased shoot length at Day 0 compared to the control. Formula 2 OD₆₀₀=1 increased shoot length at Day 0, 2 and Day 0, 4 compared to the control.

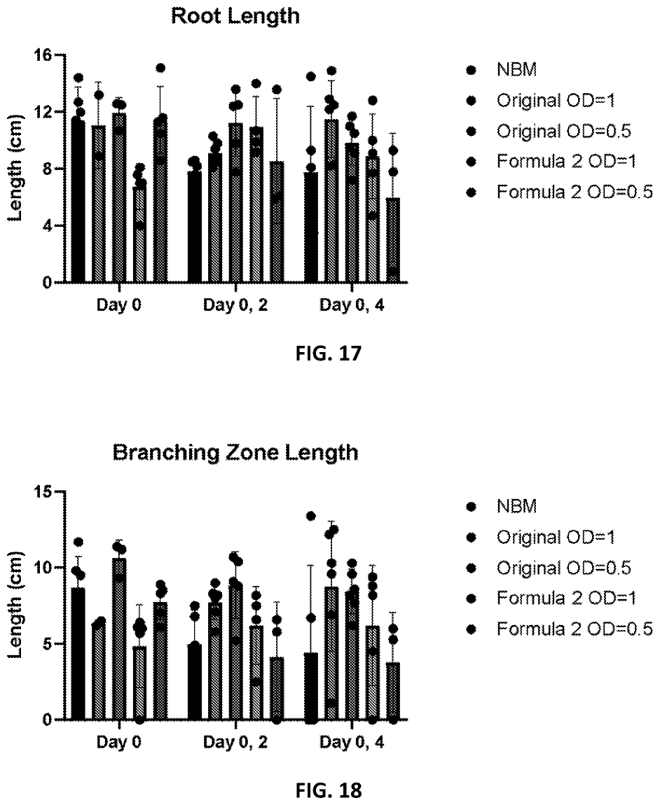

The Original inoculant increased root length in hemp seedlings when applied twice to the soil (FIG. 17). Each solid bar represents the average root length with individual values (n≥2). Measurements were taken 7 days post-inoculation. At Day 0, both original inoculants and Formula 2 OD₆₀₀=0.5 were comparable to the NBM control. At Day 0, 2, both Original inoculants and Formula 2 OD₆₀₀=1 resulted in an increase in root length when compared to the control. At Day 0, 4, both Original inoculants and Formula 2 OD₆₀₀=1 resulted in an increase in root length when compared to the control.

The Original OD₆₀₀=0.5 inoculant increased branching zone length in hemp seedlings across all application times (FIG. 18). Each solid bar represents the average shoot length with individual values (n≥2). Measurements were taken 7 days post-inoculation. At Day 0, the Original OD₆₀₀=0.5 inoculant resulted in an increased in branching zone length compared to the NBM control. Both Original inoculants and Formula 2 OD₆₀₀=1 resulted in an increase in branching zone length at Day 0, 2 and Day 0, 4 compared to the controls.

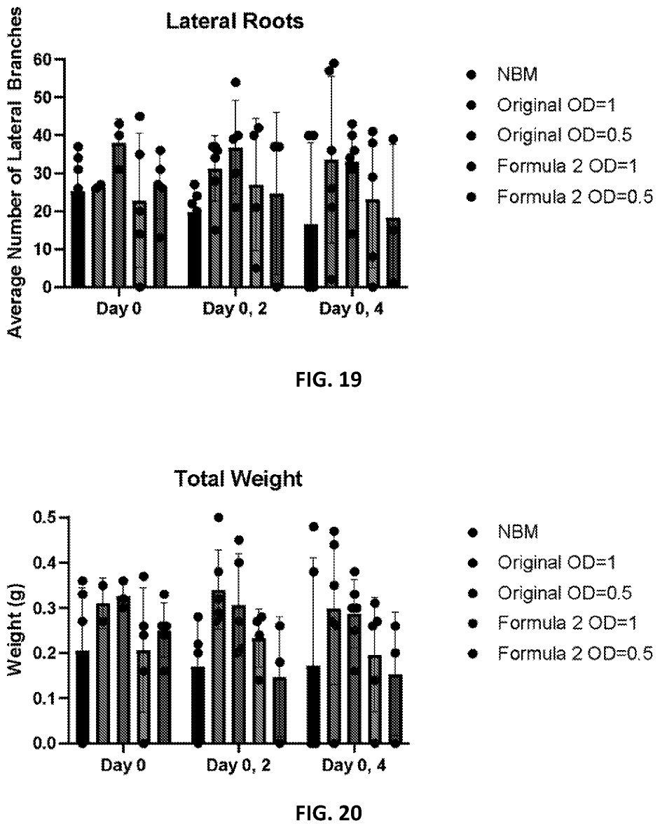

The Original inoculant at OD₆₀₀=1 increased the number of lateral roots in hemp seedlings across all application times (FIG. 19). Each solid bar represents the average number of lateral root branches with individual values (n≥2). Measurements were taken 7 days post-inoculation. The Original inoculant at OD₆₀₀=1 increased the number of lateral branches at all application times compared to the NBM control. At Day 0, 2, Original inoculant at OD₆₀₀=0.5 and both Formula 2 inoculants increased the number of lateral branches compared to the control. At Day 0, 4, the Original inoculant OD₆₀₀=0.5 and Formula 2 OD₆₀₀=1 increased the number of lateral branches compared to the control group.

The Original inoculant increased total hemp seedling weight across all application times (FIG. 20). Each solid bar represents the average total weight of hemp seedlings with individual values (n≥2). Measurements were taken 7 days post-inoculation. Both Original inoculants increased the total seedling weight compared to the NBM control group at all tree application times. Formula 2 OD₆₀₀=0.5 increased total seedling weight at Day 0 and Formula 2 OD₆₀₀=1 increased total seedling weight at Day 0, 2 compared to the control.

The Original inoculant increased root weight in hemp seedlings across all application times (FIG. 21). Each solid bar represents the average shoot length with individual values (n≥2). Measurements were taken 7 days post-inoculation. The Original inoculants increased seedling root weight at all three application times compared to the NBM control. Formula 2 OD₆₀₀=1 increased root weight at Day 0, 2 compared to the control.

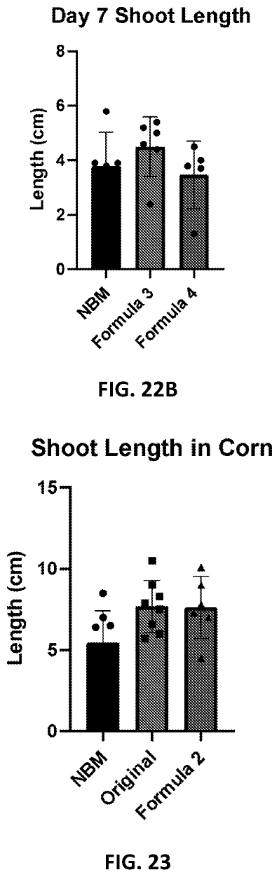

Formula 3 increased shoot length at day 7 post-inoculation in hemp seedlings (FIGS. 22A and 22B). Each solid bar represents the average shoot length with individual values (n≥2). Seeds were sown in soil, then inoculant was added on the soil surface directly above the seed. Measurements were taken 4 days and 7 days post-inoculation. At Day 4, Formula 4 increased shoot length compared to the NBM control (FIG. 22A). At Day 7, Formula 3 increased shoot length compared to the control group (FIG. 22B).

### Example 7: Treatment of Corn Seeds with Rooting Inoculants

Preparation and composition of microbial inoculants was as described in Example 6. Corn seeds were planted in soil and inoculated on the soil surface at planting. Measurements were taken 7 days after planting.

Sweet corn seedlings treated with the rooting inoculants had taller shoots compared to the NBM control (FIG. 23). Each solid bar represents the average shoot length with individual values (n≥6).

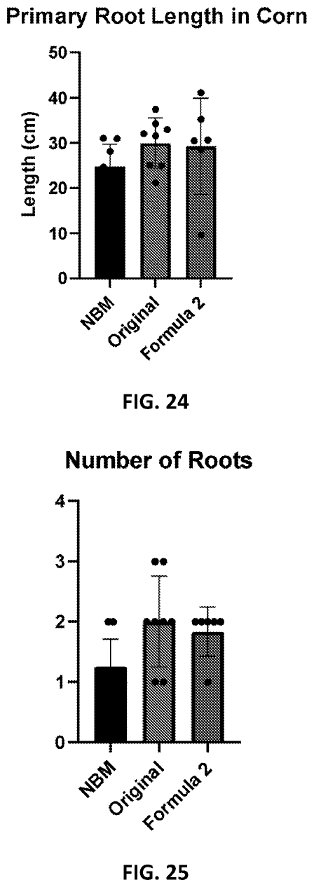

Sweet corn seedlings treated with the rooting inoculants had longer primary roots compared to the NBM control (FIG. 24). Each solid bar represents the average primary root length with individual values (n≥6).

Sweet corn seedlings treated with the rooting inoculants had more roots compared to the NBM control (FIG. 25). Each solid bar represents the average number of primary and seminal roots combined with individual values (n≥6).

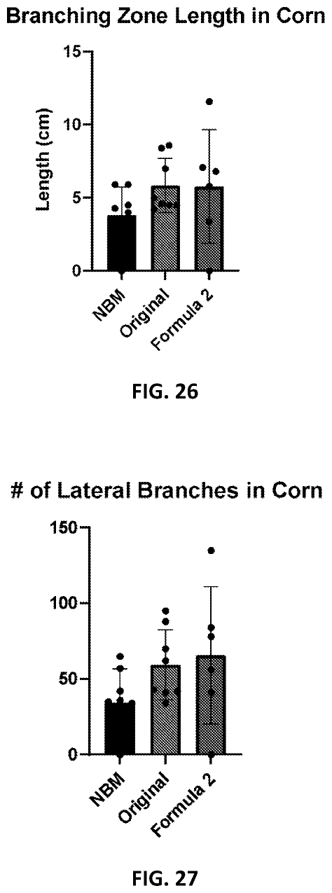

Sweet corn seedlings treated with the rooting inoculants had longer branching zones compared to the NBM control (FIG. 26). Each solid bar represents the average branching zone length with individual values (n≥6).

Sweet corn seedlings treated with the rooting inoculants had more lateral branches on the primary root compared to the NBM control (FIG. 27). Each solid bar represents the average number of lateral branches with individual values (n≥6).

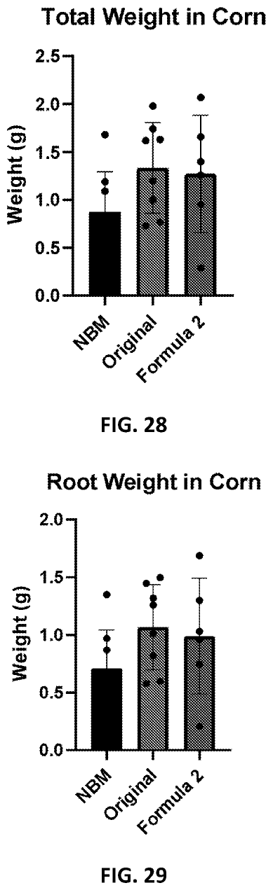

Sweet corn seedlings treated with the rooting inoculants weighed more than the NBM control (FIG. 28). Each solid bar represents the average total seedling weight with individual values (n≥6).

Sweet corn seedlings treated with the rooting inoculants had greater root weights compared to the NBM control (FIG. 29). Each solid bar represents the average seedling root weight with individual values (n≥6).

### Example 8: Indole Acetic Acid Levels in Rooting Inoculants Supplemented with Tryptophan

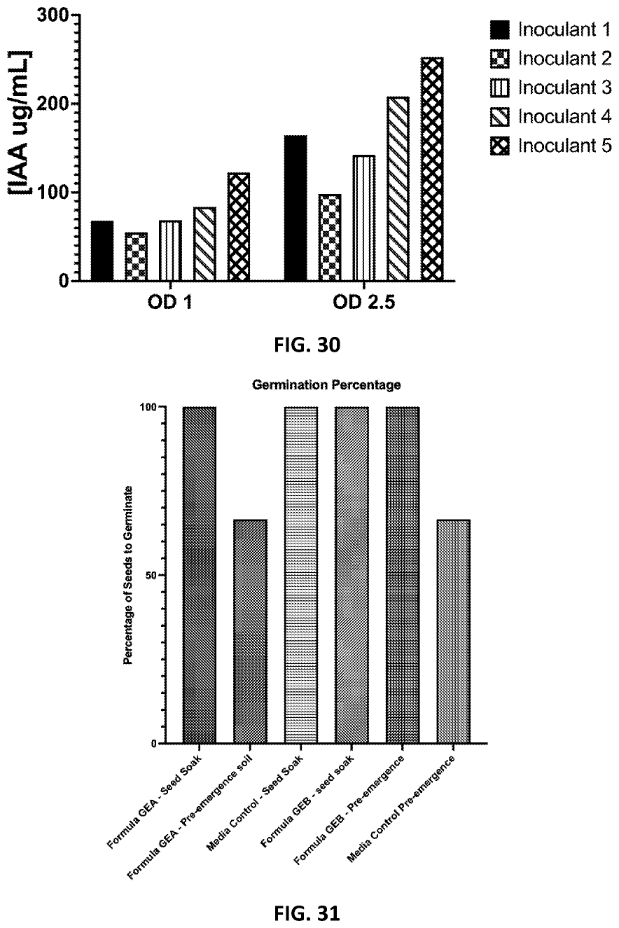

Indole acetic acid (IAA) levels were measured in rooting microbial inoculants supplemented with tryptophan using the Salkowski method (17). The rooting inoculants were comprised of the following microbial species. Inoculant 1: G. diazotrophicus, A brasilense, P. putida, and T. virens; Inoculant 2: G. diazotrophicus, P. fluorescens, A. brasilense, P. putida, and T virens; Inoculant 3: G. diazotrophicus, P. fluorescens, A. brasilense, P. putida; Inoculant 4: G. diazotrophicus, A. brasilense, P. putida; Inoculant 5: G. diazotrophicus, and P. fluorescens. The results are shown in FIG. 30.

### Example 9: Germination Inoculants

Preparation of microbial inoculants was as described in Example 4. The germination inoculants were comprised of the following microbial species. Formula GEA: A. brasilense, P. putida, and A. chroococcum; Formula GEB: A. brasilense, P. putida, A. chroococcum, and B. mobilis.

### 9.1 Corn Germination

Corn seeds were planted and either treated immediately with a 10-mL soil application of inoculant (pre-emergence soil; FIG. 31) or soaked in tubes filled with the appropriate formulation for one hour and then planted in the soil (seed soak; FIG. 31). FIG. 31 shows the germination percentage after 4 days. The seed soak treatments were the most effective germination enhancers of corn seeds. Each bar represents the average germination percentage of the corn.

### 9.2 Cosmos Germination

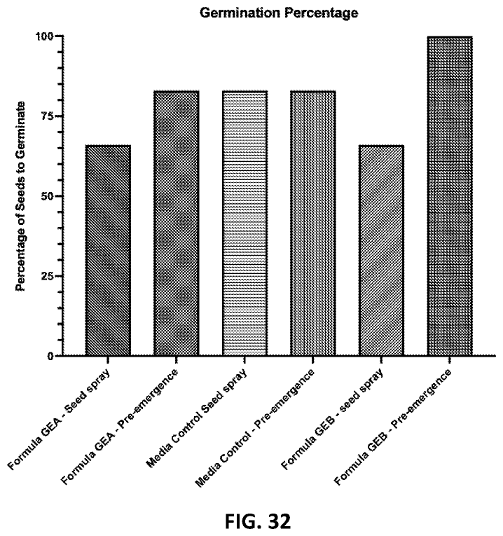

Cosmos seeds were planted and either treated immediately with a 10 mL soil application (pre-emergence; FIG. 32), or sprayed with inoculant, air dried, and then planted (seed spray; FIG. 32). FIG. 32 shows the germination percentage after 6 days. Formula GEB applied pre-emergence was the most effective germination enhancer. Each bar represents the average germination percentage of the cosmos.

### 9.3 Legume Germination

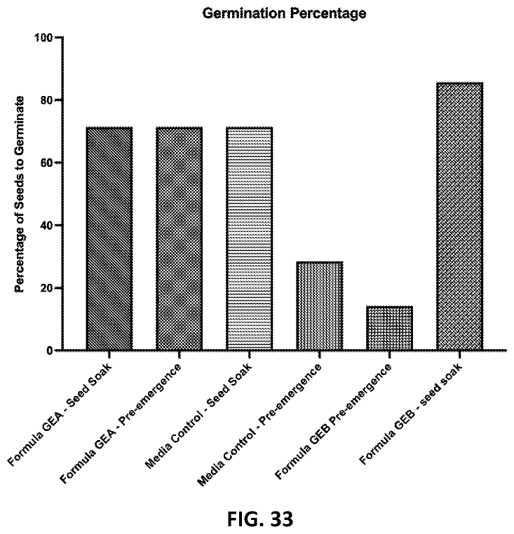

Legume seeds were treated as described in Example 8.1. FIG. 33 shows the germination percentage after 7 days. Formula GEB seed soak treatment was the most effective germination enhancer. Each bar represents the average germination percentage of the legumes.

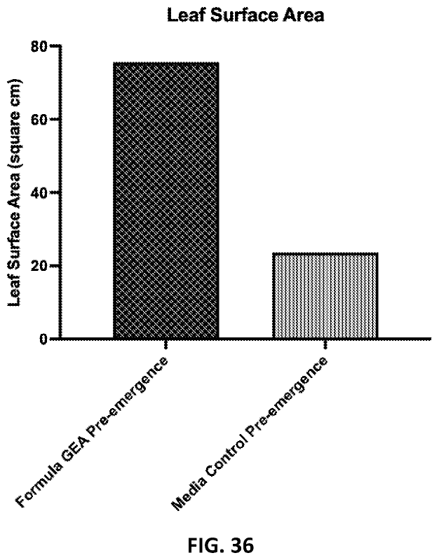

Formula GEA applied before germination (pre-emergence) was more effective at increasing legume leaf surface area compared to the media control (FIG. 36). Each bar represents the average leaf surface area of the legumes. Measurements were taken 10 days post-germination using the imaging and analytical software ImageJ.

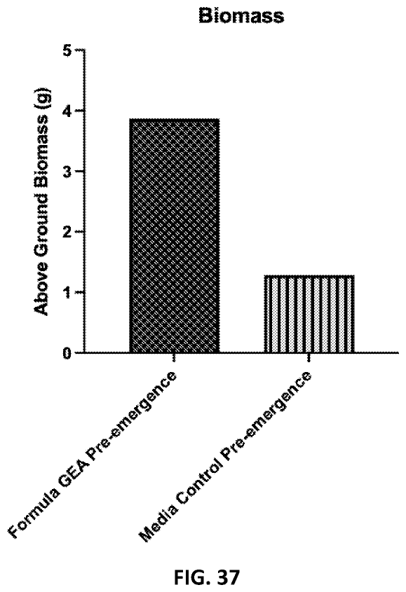

Formula GEA applied before germination (pre-emergence) was more effective at increasing legume biomass compared to the media control (FIG. 37). Each bar represents the average biomass of the legumes. Measurements were taken 10 days post germination using an analytical scale.

### 9.4 Sunflower Germination

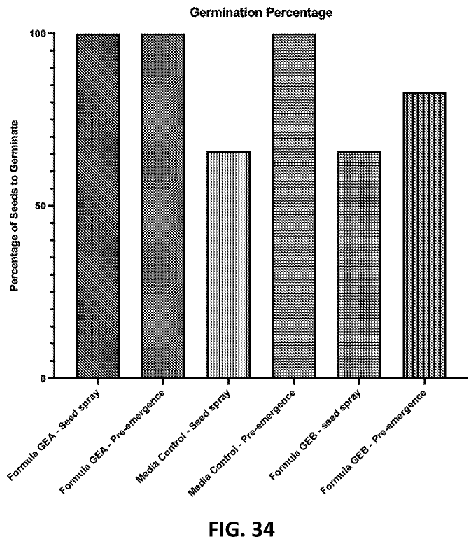

Sunflower seeds were treated as described in Example 8.2. FIG. 34 shows the germination percentage after 6 days. Formula GEA regardless of application method was the best germination enhancer. Each bar represents the average germination percentage of the cosmos.

### 9.5 Hemp Germination Biomass

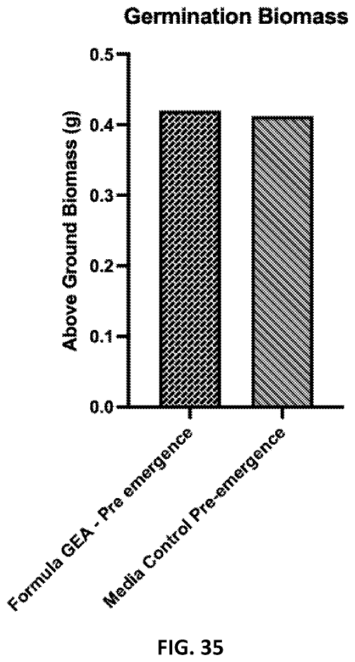

Hemp seeds were planted and treated immediately with a 10 mL soil application. Formula GEA applied before germination (pre-emergence) was more effective at increasing biomass compared to the media control (FIG. 35). Each bar represents the average biomass of the hemp. Measurements were taken after 14 days.

### REFERENCES

1. IBIS. 2019. Fertilizer Manufacturing Industry in the U.S.
2. Citation Daniel J Sobota et al 2015 Environ. Res. Lett. 10 025006
3. Foucault, A., Vallet, N., Ravalet, N. et al. Occupational pesticide exposure increases risk of acute myeloid leukemia: a meta-analysis of case-control studies including 3,955 cases and 9,948 controls. Sci Rep 11, 2007 (2021). https://doi.org/10.1038/s41598-021-81604
4. Agricultural chemical exposures and birth defects in the Eastern Cape Province, South Africa A case-control study. Gudrun A Heeren, Joanne Tyler, Andrew Mandeya. Environ Health. 2003; 2: 11. Published online 2003 Oct 4. doi: 10.1186/1476-069X-2-11
5. Christopher W. Tessum et al., “Inequity in consumption of goods and services adds to racial-ethnic disparities in air pollution exposure,” Proceedings of the National Academy of Sciences (March 2019).
6. C. J. Bronick, R. Lal, Soil structure and management: a review, Geoderma, Volume 124, Issues 1-2, 2005, Pages 3-22, ISSN 0016-7061, https://doi.org/10.1016/j.geoderma.2004.03.005.
7. Herring, Peg. “The Secret Life of Soil.” OSU Extension Service, Oregon State University Extension Service, Jan. 2010, extension.oregonstate.edu/news/secret-life-soil.
8. Ingham, Elaine R. “Soil Bacteria.” Natural Resources Conservation Service Soils, United States Department of Agriculture, www.nrcs.usda.gov/wps/portal/nrcs/detailfull/soils/health/biology/?cid=nrcs142p2_053862.
9. Ingham, Elaine R. “Soil Fungi.” Natural Resources Conservation Service Soils, United States Department of Agriculture, https://www.nrcs.usda.gov/wps/portal/nrcs/detailfull/soils/health/biology/?cid=nrcs142p2_05 3864.
10. McNear Jr., D. H. (2013) The Rhizosphere-Roots, Soil and Everything In Between. Nature Education Knowledge 4(3):1
11. J. Handelsman, E. V. Stabb. The Plant Cell Oct 1996, 8 (10) 1855-1869; DOI: 10.1105/tpc.8.10.1855
12. Fabricio Cassan, Martin Diaz-Zorita, Azospirillum sp. in current agriculture: From the laboratory to the field, Soil Biology and Biochemistry, Volume 103, 2016, Pages 117-130, ISSN 0038-0717, https://doi.org/10.1016/j.soilbio.2016.08.020.
13. Cope, Kevin R et al. “The Ectomycorrhizal Fungus Laccaria bicolor Produces Lipochitooligosaccharides and Uses the Common Symbiosis Pathway to Colonize Populus Roots.” The Plant cell vol. 31,10 (2019): 2386-2410. doi:10.1105/tpc.18.00676
14. Crossay, T., Majorel, C., Redecker, D. et al. Is a mixture of arbuscular mycorrhizal fungi better for plant growth than single species inoculants? Mycorhize 29, 325-339 (2019). https://doi.org/10.1007/s00572-019-00898-y
15. Hemlata Chauhan, D.J. Bagyaraj, G. Selvakumar, S.P. Sundaram, Novel plant growth promoting rhizobacteria-Prospects and potential, Applied Soil Ecology, Volume 95, 2015, Pages 38-53, ISSN 0929-1393, https://doi.org/10.1016/j.apsoil.2015.05.011.
16. Gao, X., Guo, H., Zhang, Q. et al. Arbuscular mycorrhizal fungi (AMF) enhanced the growth, yield, fiber quality and phosphorus regulation in upland cotton (Gossypium hirsutum L.). Sci Rep 10, 2084 (2020). https://doi.org/10.1038/s41598-020-59180-3.
17. Gilbert S, Xu J, Acosta K, Poulev A, Lebeis S and Lam E (2018) Bacterial Production of Indole Related Compounds Reveals Their Role in Association Between Duckweeds and Endophytes. Front. Chem. 6:265. doi: 10.3389/fchem.2018.00265

1. A composition for promoting plant rooting or seed germination, comprising a mixture of:
at least one first microbial species selected from Azospirillum brasilense, Sphingomonas paucimobilis, Rhizophagus irregularis, Bradyrhizobium japonicum, Paenibacillus mucilaginosus, Pseudomonas putida, Variovorax paradoxus, Rhizophagus intraradices, Rhizophagus diaphanus, Rhizophagus clarus, Saccharomyces cerevisiae, and Trichoderma harzianum; and
at least one second microbial species selected from Pseudomonas fluorescens, Azotobacter chroococcum, Herbaspirillum seropedicae, Paenibacillus lentimorbus, Azorhizobium caulinodans, Gluconacetobacter diazotrophicus, Trichoderma hamatum, Trichoderma virens, Beijerinckia mobilis, and Laccaria bicolor.

2. The composition of claim 1, wherein the at least one first microbial species is selected from Azospirillum brasilense, Rhizophagus irregularis, Paenibacillus mucilaginosus, Pseudomonas putida, and Rhizophagus intraradices, and the at least one second microbial species is selected from Azotobacter chroococcum, Herbaspirillum seropedicae, Paenibacillus lentimorbus, Beijerinckia mobilis, and Trichoderma hamatum.

3. The composition of claim 1, wherein the composition promotes rooting capacity, wherein the composition comprises:
a) A. brasilense, P. putida, G. diazotrophicus, T. virens, and P. fluorescens;
b) A. brasilense, P. putida, G. diazotrophicus, T. virens, P. fluorescens, and S. cerevisiae;
c) P. putida, A. brasilense, P. lentimorbus, and T. virens;
d) G. diazotrophicus, A. caulinodans, P. fluorescens, V. paradoxus, and T. virens; or
e) P. putida, A. brasilense, P. lentimorbus, T. virens, G. diazotrophicus, A. caulinodans, P. fluorescens, and V. paradoxus.

4. The composition of claim 1, wherein the composition promotes seed germination, wherein the composition comprises:
a) A. brasilense, P. putida, and A. chroococcum; or
b) A. brasilense, P. putida, A. chroococcum, and B. mobilis.

5. The composition of any one of claim 1, wherein the microbial species are lyophilized.

6. A microbial inoculant comprising:
a composition of claim 1; and
water or an aqueous solution.

7. The microbial inoculant of claim 6, further comprising a carbon source.

8. The microbial inoculant of claim 7, wherein the carbon source is a hexose.

9. The microbial inoculant of claim 8, wherein the hexose is glucose.

10. The microbial inoculant of claim 7, wherein the percent weight of the carbon source in the total volume of the microbial inoculant ranges from 9-15%, 10-14%, or 11-13% (w/v).

11. The microbial inoculant of claim 6, further comprising a thickening agent.

12. The microbial inoculant of claim 11, wherein the thickening agent is aloe vera, aloe vera flakes, willow bark extract, silica, pectin, or psyllium husk.

13. The microbial inoculant of claim 11, wherein the percent weight of the thickening agent in the total volume of the microbial inoculant ranges from 2-8%, 3-7%, or 4-6% (w/v).

14. A method for promoting plant rooting comprising contacting an ungerminated seed, a germinated seed upon planting, a seedling or roots at transplanting, a cutting at propagation, a maturing young plant after planting, or a maturing plant at a time before or at the time of flowering, with a with a microbial inoculant of claim 5.

15. A method for promoting seed germination comprising contacting an ungerminated seed, a germinated seed upon planting, a seedling or roots at transplanting, a cutting at propagation, a maturing young plant after planting, or a maturing plant at a time before or at the time of flowering, with a with a microbial inoculant of claim 5.

16. The method of claim 14, wherein the contacting with a mixture of microbial species occurs seasonally, bimonthly, monthly, every 3 weeks, every 2 weeks, weekly, every 5 days, every 3 days, daily, or a similar such period of time appropriate for the grow space and plant species.

17. The method of claim 15, wherein the contacting with a mixture of microbial species occurs seasonally, bimonthly, monthly, every 3 weeks, every 2 weeks, weekly, every 5 days, every 3 days, daily, or a similar such period of time appropriate for the grow space and plant species.
